(19) United States
(12) Patent Application Publication
(10) Pub. No.: US 2004/0036855 A1
(43) Pub. Date: Feb. 26, 2004

(54) Process and apparatus for measuring the concentration of oil in water

(76) Inventors: Erling Hammer, Mjolkeraen (NO); Erik Mucunguzi, Kyambogo (UG); Eirik Abro, Nesttun (NO)

Correspondence Address: CARELLA, BYRNE, BAIN, GILFILLAN, CECCHI,, STEWART & OLSTEIN, 6 BECKER FARM ROAD, ROSELAND, NJ, 07068, US

(21) Appl. No.: 10/362,390

(22) PCT Filed: Sep. 3, 2001
(86) PCT No.: PCT/NO01/00364

(30) Foreign Application Priority Data
Sep. 1, 2000 (NO) 20004341

(51) Int. Cl. G01N 33/28; G01J 4/00; G01N 21/85

(52) U.S. Cl. 356/70; 356/364

(57) ABSTRACT

It is disclosed a device and a method for the on-line determination of the concentration of oil in a water solution by the angle of rotation of plane polarized light.

The present invention relates to a device and a method for the on-line determination of minor amount of oil in process water.

When a beam of plane polarized light is passed through a translucent medium it encounters a number of optical phenomena like scattering, absorption, and optical rotation of the polarization plane.

If a beam of linearly polarized light is directed through a liquid, the plane of polarization is gradually rotated about the optical axis in the liquid.

This phenomenon of rotation of the plane of polarization is called optical activity. Liquids made up of optically active substances and inactive solvents are found to produce a rotation proportional to the amount of active substances present. The rotation is nearly proportional to the inverse square of the wavelength of the polarized light.

Substanses that rotate the plane of polarization clock wise, looking back towards the source, are called dextrorotatory. Those that create rotation counter clockwise are called levorotatory. Most of the liquids known to exhibit optical rotation are organic compounds involving complex molecules, such as cinnabar, sodium clorate, sugar solution and crystals.

Examples from the prior art which are known to the applicant, and which relate to the use of polarized light in performing a spectroscopic analyses of a specimen, include the following:

U.S. Pat. No. 3.724.952 describes an apparatus for polarimetric analyses of a specimen, comprising the use of light that is polarized in one plane. When the polarized light has passed through the specimen, the polarization shift is determined.

U.S. Pat. No. 5,009,230 describes a device for non-invasive determination of blood glucose of a patient based upon the effect of glucose in rotating polarized infrared light. Two orthogonal and equal polarized states of infrared light of minimal absorption are passed through the specimen and a determination of change in signal intensity is made due to the angle of rotation of these states.

The purpose of the present invention is to detect minor amounts of oil in an aqueous solvent, e.g. water, and preferable the sensitivity of the device and method must be sufficient to detect amounts of the compound as low as 10 ppm.

WO 00/60350 discloses a non-invasive apparatus and method for optically sensing the glucose concentration of a solution, based on the magnetic optical rotary effect (MORE) of glucose.

EP 805 352 A2 discribes a method and apparatus for urinalyis by examining the concentration of glucose and protein in the urine by measuring the angle of rotation of a urine sample.

It is thus known that the optical rotation of compounds such as glucose and proteins can be used to determine the concentration of said compounds in solvent system.

It is also known that if a d.c. magnetic field is applied parallel to, and in the same direction as the polarized beam the rotation angel will increase. This is known as induced-circular birefringence and often called the Farady effect. The magnitude of the angle of rotation is proportional to the magnetic induction, as given by the equation 1:

θ=VHL cos φ

where V=Verdet constant in rad T⁻¹ m⁻¹, H=magnetic induction (A/m), L=path length and φ is the angle between the direction of the light beam and the magnetic field.

We have now surprisingly found that the method of measuring the angle of rotation also can be used to determine the amount of an oil fraction in a solvent such as water.

Process water from the oil industry contains fractions of oil components. Environmental regulations states that process water must not exceed 40 mg/l (40 ppm) of oil. It is thus a purpose of the present invention to provide an apparatus that on-line can monitor the concentration of oil in the process water.

The process water is transported in pipelines with a diameter typically of 4 inches. As far as we know there is not available any method and apparatus for such an on-line monitoring. The amount of oil in the process water is today measured by taking out samples, for instance every day, and then conducting a chemical analysis of these samples.

We have now shown that oil fractions of about 10 ppm can be measured by the use of optical rotation, and the present invention thus provides a very sensitive, real-time and on-line monitoring of the oil concentration of such process water.

These results have been obtained without the use of a magnetic field, and it is thus anticipated that the sensitivity of the method and apparatus in accordance with the invention can be improved with such a field, as it is known that the magnitude of the polarization angles increases if a magnetic field is arranged parallel to the direction of the propagation of the light.

It is also known that the angle of rotation depends on the temperature of the sample, and a preferable embodiment of the apparatus thus contains means for sensing the temperature of the process water.

The present invention will now be further described with reference to the accompanying figures.

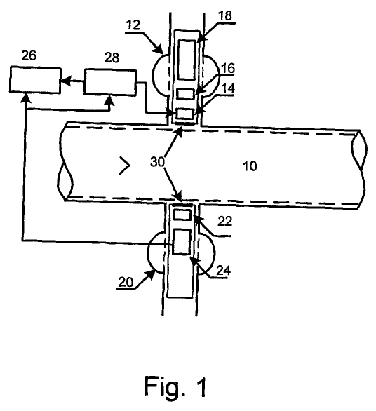

FIG. 1 shows an apparatus in accordance with the present invention.

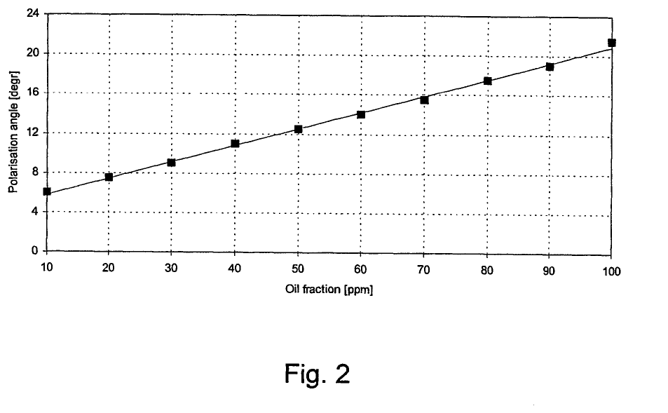

FIG. 2 shows the measured polarisation angle versus fractions of crude oil in process water at room temperature.

FIG. 1 shows a flow pipe 10 transporting process water. This process water contains small fractions of oil contamination, normally seawater with about 0-100 ppm of oil. The pipeline 10 is in a section equipped with two optical windows 30, such that at light beam can transverse the process water flowing through the pipe line 10. A light source 18, such as a laser, emits light, and this light passes through a polarization filter 16 and through the window 30 and into the process water. The angle of rotation is measured for the light which has passed through the process water, the window 30 and the polarization filter 22.

Optionally, the apparatus also contains a Faraday rotator 14. The apparatus also contains two valves, 18 and 20. These valves are needed for disconnecting the apparatus from the process water flow for cleaning of the optical windows and for maintenance.

The apparatus also contains a data acquisition unit 26 and a control unit 28. The control unit is used to control the Faraday rotator that rotates the polarization of the light. The rotation range from 0° to 90° dependent on the input voltage. Based on the transmitted light measured by the photo detector, the control unit sets the current to the Faraday rotator in such a way that minimum (or maximum) light intensity is detected.

The rotation angle generated by the Faraday rotator gives the optical polarization angle in the process water.

The rotation angle decreases for increasing wavelength of the light. In the measurements presented in FIG. 2, a green laser (λ=543 nm) was used. A broad beam laser can be used to minimize the effect of inhomogeneous distribution of the oil droplets.

To our knowledge, the applicants of the present invention have for the first time showed that optical rotation can be used for the determination of minor amounts of oil in a water solvent. Further, it has been shown that these measurements are sensitive enough to detect oil fractions as low as 10 ppm, and this can thus be used as an environmental monitor of process water where the amount of oil must not exceed 40 ppm (according to international regulations). It has also been shown that the apparatus in accordance with the invention provides a non-intrusive method for on-line, real-time monitoring of small amounts of oil in such process water.

### EXAMPLE 1

Determination of Minor Amounts of Oil in a Water Solvent

The FIG. 2 shows the linear relationship between the concentration of oil in water and the polarization angle. This change in angle of rotation (θ) can therefore be used to detect contaminations of oils in the water with sensitivity better than 10 ppm (parts per million).

1. An apparatus for the on-line determination of fractions of oil in process water in a pipeline, characterized in that the pipeline is equipped with two opposite arranged optical windows which allows for the passage of a light beam through the process water in the pipeline, and that the apparatus comprises a light source (18) and a polarization filter (16) which passes plane polarized light through the process water in the pipeline, and a polarizing filter (22) and a photo detector (24) for measuring the rotation angle of the polarization plane of the light which has propagated through the liquid.

2. An apparatus in accordance with claim 1, characterized in that the apparatus further comprises a data acquisition unit 26 and a control unit 28.

3. An apparatus is accordance with claim 1, characterized in that it further comprises a Faraday rotator 14.

4. An apparatus in accordance with claim 3, characterized in that the control unit 28 is used to control the Faraday rotator.

5. An apparatus in accordance with claim 1, characterized in that the apparatus comprises a temperature sensor.

6. Device in accordance with one of the claims 1-5, characterized in that the light source is a laser.

7. Device in accordance with claim 6, characterized in that the laser emits green light.

8. A method for the determination of the concentration of oil fraction in an aqueous solvent, characterized in that an optically active component in the oil fraction is determined by measuring an angle of rotation of said oil fraction.

9. A method in accordance with claim 8, characterized in that the determination of the concentration of oil in the aqueous solvent is conducted on-line, i.e. as the aqueous solvent passes through a pipeline.

10. A method in accordance with claim 8, characterized in that optical windows are arranged in the pipeline, enabling light to passing through the aqueous solvent.

11. A method in accordance with claim 8, characterized in that a light source (18) passes light through a polarization filter (16), thus generating plane polarized light which is passed through the solvent in the pipeline, and where angle of rotation of the propagated light is detected by the means of a polarization filter (22) and a photo detector (24).

12. A method in accordance with claim 11, characterized in that signals from the photo detector (24) is transmitted to the data acquisition unit (26) and a control unit (28) for processing.

13. A method in accordance with claim 12, characterized in that the control unit controls a Faraday regulator arranged upstream of the polarization filter (16).

14. A method in accordance with one of the claims 8-13, characterized in that a temperature sensor determines the temperature of the aqueous solvent, and that information from this sensor is communicated to the data acquisition and analysis unit to compensate for temperature variations.

15. A method in accordance with one of the claims 8-14, wherein the angle of rotation of plane polarized light is determined at various magnetic field strengths.

16. Use of an apparatus in accordance with one of the claims 1-7, or a method in accordance with one of the claims 8-15 for the determination of the concentration of oil in a water solution.

17. Use in accordance with claim 16, characterized in that said water solution is process water.

18. Use in accordance with claim 17, characterized in that said process water is process water from the oil industry.
